(19) United States
(12) Patent Application Publication
(10) Pub. No.: US 2002/0197253 A1
(43) Pub. Date: Dec. 26, 2002

(54) Compositions and methods for promoting or inhibiting NDPK

(76) Inventors: Dennis J. Cheek, Cary, NC (US); Salvatore V. Pizzo, Bahama, NC (US); Tammy L. Moser, Chapel Hill, NC (US)

Correspondence Address: WOMBLE CARLYLE SANDRIDGE & RICE, PLLC, Post Office Box 7037, Atlanta, GA, 30357-0037, US

(21) Appl. No.: 10/153,153

(22) Filed: May 22, 2002

Related U.S. Application Data
(60) Provisional application No. 60/292,577, filed on May 22, 2001.

(51) Int. Cl. A61K 39/395

(52) U.S. Cl. 424/141.1; 424/146.1; 514/12

(57) ABSTRACT

Compounds, compositions and methods for promoting or inhibiting angiogenesis, and screening methods for identifying compounds are disclosed. The compounds bind to NDPK or angiostatin. When bound to these NDPK, they can function as angiogenesis inhibitors. When bound to angiostatin in a manner that inhibits the ability of angiostatin to bind to NDPK, they can function as angiogenesis promoters. The compounds can be, for example, antibodies, antibody fragments, enzymes, peptides, nucleic acids such as oligonucleotides, or small molecules. The antibodies can be monoclonal, humanized, or polyclonal antibodies. The compounds can be conjugated to or combined with various cytotoxic agents and/or labeled compounds. Methods for promoting angiogenesis can be used to introduce vasculature to areas in a patient that can benefit from such increased vasculature. Methods for inhibiting angiogenesis can be used to treat disorders mediated by angiogenesis, for example, tumors, autoimmune disorders such as rheumatoid arthritis, and the like.

### RELATED APPLICATION

This application is a non-provisional application claiming the benefit of Provisional Application Serial No. 60/292,577, filed May 22, 2001, the content of which is hereby incorporated in its entirety.

### FIELD OF THE INVENTION

This application is generally in the area of compositions and methods for promoting or inhibiting NDPK, and, accordingly, promoting or inhibiting angiogenesis.

### BACKGROUND OF THE INVENTION

In 1953, an enzyme, appropriately named “nucleoside diphosphate kinase” (NDPK) was found in yeast by Krebs and Hems (1953) and in pigeon breast muscle by Berg and Jolic (1953). The enzyme is ubiquitously distributed in animals, plants and microorganisms. It has long been believed that NDPK plays a cytoplasmic “housekeeping” role in all cells as a major component of the enzymatic pathway for the synthesis of triphosphate nucleotides. NDPK catalyzes transphosphorylation of a terminal phosphate group from a nucleoside triphosphate to a nucleoside diphosphate through formation of a high energy phosphorylated enzyme intermediate. The enzyme is capable of using either adenyl or guanyl purine nucleosides as both substrate and/or donor.

In the last two decades there has been interest in the possibility that NDPK could be involved in other cellular events in addition to intracellular generation of triphosphate nucleotides. In Myxococcus xanthus, a gram negative bacterium, NDPK has been shown to be essential for cell growth (Dorado et al., 1990). In Drosophila mlanogaster, the abnormal wing disc (awd) gene codes for a single 0.8 kb mRNA that is translated into a 17.5 kDa polypeptide, an NDPK, which is required to obtain normal wing discs (Biggs et al., 1990) and is associated with microtubules in which it plays a crucial role in spindle microtubule polymerization, probably by transphosphorylation of tubulin bound GDP (Biggs et al., 1990). An NDPK has been reported to be involved in GTP-dependent receptor-signal transduction (Kimura and Shimade, 1998; Seifert et al, 1988) and while this notion has received both support (Randazzo et all, 1991) and criticism (Randazzo et al., 1992) in recent years, the possibility that GTP production is compartmented in the region of GTP binding proteins is intriguing and suggests that NDPK may play more than a housekeeping role in mammalian cells. Two genes that encode proteins with NDPK activity have been found in humans (Giles et al., 1991).

An ecto-NDPK present on the cell surface has been suggested in tumor cell lines (Gutensohn and Riger, 1984, 1986; Ohtsuke et al., 1986,; 1987; Urano et al., 1992). In 1988, Kimura and Shimade described a membrane-associated NDPK in rat liver. Also, in 1988, the gene that expressed NDPK was initially identified by difference hybridization between low and high metastatic murine melanoma lines (Steeg et al., 1988). This gene was identified as nm23. Subsequently, high homology between NM23 (the use of capital NM is meant to refer to the protein while the use of lower case nm refers to genetic material) proteins and NDPK have been identified in a number of species, including humans (Kikkawa et al., 1990; Kimura et al., 1990; Dorado et al., 1990; Hama et al., 1991; Gilles et al., 1991). The role of NDPK in angiogenesis was not described in the prior art.

Angiogenesis is the formation of new capillary blood vessels leading to neovascularization. Angiogenesis is a complex process which includes a series of sequential steps including endothelial cell-mediated degradation of vascular basement membrane and interstitial matrices, migration of endothelial cells, proliferation of endothelial cells, and formation of capillary loops by endothelial cells.

Both controlled and uncontrolled angiogenesis are thought to proceed in a similar manner. Endothelial cells and pericytes, surrounded by a basement membrane, form capillary blood vessels. Angiogenesis begins with the erosion of the basement membrane by enzymes released by endothelial cells and leukocytes. The endothelial cells, which line the lumen of blood vessels, then protrude through the basement membrane.

Angiogenic stimulants induce the endothelial cells to migrate through the eroded basement membrane. The migrating cells form a “sprout” off the parent blood vessel, where the endothelial cells undergo mitosis and proliferate. The endothelial sprouts merge with each other to form capillary loops, thereby creating the new blood vessel.

In normal physiological processes such as wound healing, angiogenesis is turned off once the process is completed. In contrast, tumor angiogenesis is not self-limiting. The progressive growth of solid tumors beyond clinically occult sizes (e.g., a few mm3) requires the continuous formation of new capillary blood vessels to deliver nutrients and oxygen for the tumor itself to grow, a process known as tumor angiogenesis. Solid tumors elicit an angiogenic response in the surrounding normal tissue for further growth. The resultant neovascularization of the tumor is associated with more rapid growth, and local invasion. Therefore, either inhibition of tumor angiogenesis (antiangiogenic therapy) or selective destruction of a tumor's existing blood vessels (vascular targeting therapy) would suppress or arrest tumor growth and its spread.

Further, in certain pathological (and nonmalignant) processes, angiogenesis is abnormally prolonged. Examples include ocular neovascular disease, which is characterized by invasion of new blood vessels into the retina or cornea, as well as other eye-related diseases. Other angiogenesis-associated diseases include diabetic retinopathy and chronic inflammatory diseases such as rheumatoid arthritis, dermatitis and atherosclerosis.

Antiangiogenic therapy has been proposed for modulating such angiogenesis-associated disorders. One approach has been to administer VEGF (vascular endothelial growth factor) inhibitors. Other approaches involve using angiostatin or endostatin, which are both known to inhibit angiogenesis. The in vivo use of angiostatin or endostatin is somewhat limited by their relatively short half-lives in vivo.

It would be advantageous to have new antiangiogenic compositions and methods to add to the arsenal of therapies available for treating these angiogenesis-mediated disorders. It would also be advantageous to have new methods for identifying such compositions and methods. The present invention provides such compositions and methods.

### SUMMARY OF THE INVENTION

The present invention is directed to compounds, compositions and methods for inhibiting angiogenesis, and results from the discovery that angiostatin binds to NDPK and/or to a complex containing NDPK (hereinafter referred to as binding to NDPK), and, when so bound, inhibits angiogenesis.

Compounds useful for inhibiting angiogenesis bind to NDPK, and, in particular, to the H1 subunit of NDPK. When so bound, they inhibit the ability of NDPK to promote angiogenesis. The compounds can be, for example, angiostatin, antibodies, antibody fragments, enzymes, proteins, peptides, nucleic acids such as oligonucleotides, or small molecules. The antibodies can be, for example, monoclonal, humanized (chimeric) or polyclonal antibodies, and can be prepared, for example, using conventional techniques. The compounds can be conjugated to various cytotoxic agents and/or labeled compounds.

The compounds can be included in various compositions, for example, compositions suitable for intravenous, intramuscular, topical, local, intraperitoneal, or other forms of administration. They can be targeted to capillary beds by incorporating them into appropriately sized microparticles or liposomes that remain lodged in capillary beds and release the compounds at a desired location. The methods involve administering an effective, anti-angiogenic amount of the compounds to a patient in need of treatment thereof.

The present invention is also directed to compounds, compositions and methods for promoting angiogenesis by inhibiting the ability of angiostatin to bind to NDPK and inhibit angiogenesis. Compounds suitable for promoting angiogenesis can be, for example, antibodies, antibody fragments, enzymes, proteins, peptides, nucleic acids such as oligonucleotides, or small molecules, that are capable of binding to angiostatin.

These compounds can also be included in various compositions, and can also be targeted to capillary beds. The methods involve administering an effective, angiogenesis-promoting amount of the compounds to a patient in need of treatment thereof.

Methods for promoting angiogenesis can be used to introduce vasculature to areas in a patient that can benefit from such increased vasculature. Methods for inhibiting angiogenesis can be used to treat disorders mediated by angiogenesis, for example, tumors, autoimmune disorders such as rheumatoid arthritis, and the like. The methods involve administering effective amounts of suitable angiogenic or anti-angiogenic compounds and/or compositions including the compounds to patients in need of treatment. Effective angiogenic amounts are amounts effective to promote angiogenesis, and effective anti-angiogenic amounts are amounts effective to inhibit at least a significant amount of the angiogenesis that would otherwise occur in the absence of treatment.

Not all compounds that bind to angiostatin will do so in a way that inhibit the ability of angiostatin to bind to NDPK. Screening methods can be used to identify compounds useful in these methods. The screening methods can identify compounds that bind to NDPK or to angiostatin. Combinatorial libraries of compounds, for example, phage display peptide libraries, small molecule libraries and oligonucleotide libraries can be screened. Compounds that bind to NDPK or angiostatin, can be identified, for example, using competitive binding studies using angiostatin, or using other screening techniques known to those of skill in the art. The effect of the compounds once bound to NDPK or angiostatin thereof can be determined, for example, by evaluating the level of ATP synthesis, the proliferation of human vascular endothelial cells (HUVEC), the viability and/or growth of tumors, wound healing, MatrigelTM tube formation and corneal pocket in mouse or rat.

### DETAILED DESCRIPTION OF THE INVENTION

The following description includes the best presently contemplated mode of carrying out the invention. This description is made for the purpose of illustrating the general principles of the inventions and should not be taken in a limiting sense.

NDPK (the protein product of nm23) is located as an ecto-enzyme on or in the membranes of human umbilical venous endothelial cells (HUVEC), and directs its product into the lumen of blood vessels to play a vital role in the regulation of blood flow. Working in concert with the endothelial cell ATP release mechanism, this ecto-NDPK can generate ATP extracellularly which could induce further nucleotide release and liberation of endothelium dependent relaxing factors (Houston et al., 1987; Mathie et al., 1991) from nearby endothelial cells. While the formation of ATP from ADP that accrues as a result of breakdown of the ATP released from endothelial cells may seem to be a futile and energetically expensive reaction, in the setting of the blood vessel, such an event would tend to preserve the availability longer in time and space (further downstream at the site of other endothelial cells) thus sustaining ATP receptor-dependent events in the blood vessel lumen and beyond. Furthermore, since ecto-NDPK can use both purine and pyrimidine triphosphates as phosphoryl donors, this mechanism is believed to generate ATP from nucleotide sources such as platelet GTP and UTP.

Ecto-NDPK is inhibited by angiostatin. This has implications in both angiogenesis and also tumorgenesis.

Compounds, compositions and methods for promoting or inhibiting angiogenesis are disclosed. Also disclosed are screening methods for identifying compounds that bind to NDPK, and, in particular, to the H1 subunit of NDPK (and therefore function as angiogenesis-inhibitors) or angiostatin (and therefore function as angiogenesis promoters).

The present invention is based on the discovery that angiostatin binds to NDPK, and, through this binding, inhibits angiogenesis.

Compounds that bind to angiostatin can inhibit the ability of angiostatin to bind to NDPK, and, accordingly, block the ability of angiostatin to inhibit angiogenesis.

Definitions

The following definitions will be helpful in understanding the compositions and methods described herein.

As used herein, the term “angiogenesis” is defined as the generation of new blood vessels into a tissue or organ. The term “endothelium” means a thin layer of flat endothelial cells that lines lymph vessels, and blood vessels.

The term “angiostatin” refers to a proteolytic fragment of plasminogen, and includes at least one and preferably at least three kringles from plasminogen. Angiostatin is a potent inhibitor of angiogenesis and the growth of tumor cell metastases (O'Reilly et al., Cell 79:315-328 (1994)). All anti-angiogenic forms of angiostatin are intended to be included within the definition of angiostatin as used herein.

Angiostatin has a specific three dimensional conformation that is defined by the kringle region of the plasminogen molecule. (Robbins, K. C., “The plasminogen-plasmin enzyme system” Hemostasis and Thrombosis, Basic Principles and Practice, 2nd Edition, ed. by Colman, R. W. et al. J.B. Lippincott Company, pp. 340-357, 1987). There are five such kringle regions, which are conformationally related motifs and have substantial sequence homology in the amino terminal portion of the plasminogen molecule.

A variety of silent amino acid substitutions, additions, or deletions can be made in the above identified kringle fragments, which do not significantly alter the fragments' endothelial cell inhibiting activity. Each kringle region of the angiostatin molecule contains approximately 80 amino acids and contains 3 disulfide bonds. Anti-angiogenic angiostatin can include a varying amount of amino- or carboxy-terminal amino acids from the inter-kringle regions and may have some or all of the naturally occurring di-sulfide bonds reduced. Angiostatin may also be provided in an aggregate, non-refolded, recombinant form.

Angiostatin can be generated in vitro by limited proteolysis of plasminogen, as taught by Sottrup-Jensen et al., Progress in Chemical Fibrinolysis and Thrombolysis 3:191-209 (1978), the contents of which are hereby incorporated by reference for all purposes. This results in a 38 kDa plasminogen fragment (Va179-Pro353). Angiostatin can also be generated in vitro by reducing plasmin (Gately et al., PNAS 94:10868-10872 (1997)) and in Chinese hamster ovary and human fibrosarcoma cells (Stathakis et al., JBC 272(33) :20641-.20645 (1997)).

Angiostatin may also be produced from recombinant sources, from genetically altered cells implanted into animals, from tumors, and from cell cultures as well as other sources. Angiostatin can be isolated from body fluids including, but not limited to, serum and urine. Recombinant techniques include gene amplification from DNA sources using the polymerase chain reaction (PCR), and gene amplification from RNA sources using reverse transcriptase/PCR.

The terms “a”, “an” and “the” as used herein are defined to mean “one or more” and include the plural unless the context is inappropriate.

As employed herein, the phrase “active agent” or “active compound” refers to angiostatin agonists, antagonists, partial agonists, inverse agonists or allosteric modulators. Examples of suitable biologically active compounds/agents include antibodies, antibody fragments, enzymes, peptides, nucleic acids, and small molecules.

Unless defined otherwise, all technical and scientific terms used herein have the same meaning as commonly understood by one having ordinary skill in the art. Although other materials and methods similar or equivalent to those described herein can be used in the practice or testing of the present invention, as would be apparent to practitioners in the art, the preferred methods and materials are now described.

I. Methods of Inhibiting Angiogenesis

There are several methods for inhibiting angiogenesis. Angiogenesis can be inhibited by administering an effective amount of angiostatin or other compounds that bind NDPK (for example, antibodies, antibody fragments, and/or small molecules) to a patient in need of such treatment. The methods can be used to treat tumors, various autoimmune disorders, hereditary disorders, ocular disorders and other angiogenesis-mediated disorders.

The therapeutic and diagnostic methods described herein typically involve administering an effective amount of the compositions described herein to a patient. The exact dose to be administered will vary according to the use of the compositions and on the age, sex and condition of the patient, and can readily be determined by the treating physician. The compositions may be administered as a single dose or in a continuous manner over a period of time. Doses may be repeated as appropriate.

The compositions and methods can be used to treat angiogenesis-mediated disorders including hemangioma, solid tumors, leukemia, lymphoma, metastasis, telangiectasia, psoriasis, scleroderma, pyogenic granuloma, myocardial angiogenesis, Crohn's disease, plaque neovascularization, coronary collaterals, cerebral collaterals, arteriovenous malformations, ischemic limb angiogenesis, corneal diseases, rubeosis, neovascular glaucoma, diabetic retinopathy, retrolental fibroplasia, arthritis, diabetic neovascularization, macular degeneration, wound healing, peptic ulcer, Helicobacter related diseases, fractures, keloids, and vasculogenesis. Specific disorders that can be treated, and compounds and compositions useful in these methods, are described in more detail below.

A. Carcinomas and Other Solid Tumors

Carcinomas that can be treated using the compounds, compositions and methods described herein include, but are not limited to, colorectal carcinoma, gastric carcinoma, signet ring type, esophageal carcinoma, intestinal type, mucinous type, pancreatic carcinoma, lung carcinoma, breast carcinoma, renal carcinoma, bladder carcinoma, prostate carcinoma, testicular carcinoma, ovarian carcinoma, endometrial carcinoma, thyroid carcinoma, liver carcinoma, larynx carcinoma, mesothelioma, neuroendocrine carcinomas, neuroectodermal tumors, melanoma, gliomas, neuroblastomas, sarcomas, leiomyosarcoma, MFII, fibrosarcoma, liposarcoma, MPNT, chondrosarcoma, basal cell carcinoma, squamous cell carcinoma and lymphomas.

B. Ocular Disorders Mediated by Angiogenesis

Various ocular disorders are mediated by angiogenesis, and can be treated using the compounds, compositions and methods described herein. One example of a disease mediated by angiogenesis is ocular neovascular disease, which is characterized by invasion of new blood vessels into the structures of the eye and is the most common cause of blindness. In age-related macular degeneration, the associated visual problems are caused by an ingrowth of chorioidal capillaries through defects in Bruch's membrane with proliferation of fibrovascular tissue beneath the retinal pigment epithelium. Angiogenic damage is also associated with diabetic retinopathy, retinopathy of prematurity, corneal graft rejection, neovascular glaucoma and retrolental fibroplasia. Other diseases associated with corneal neovascularization include, but are not limited to, epidemic keratoconjunctivitis, Vitamin A deficiency, atopic keratitis, superior limbic keratitis, pterygium keratitis sicca, periphigoid radial keratotomy, and corneal graph rejection.

Diseases associated with retinal/choroidal neovascularization include, but are not limited to, diabetic retinopathy, macular degeneration, presumed myopia, optic pits, chronic retinal detachment, hyperviscosity syndromes, trauma and post-laser complications. Other diseases include, but are not limited to, diseases associated with rubeosis (neovascularization of the angle) and diseases caused by the abnormal proliferation of fibrovascular or fibrous tissue including all forms of proliferative vitreoretinopathy.

C. Inflammation

The methods described herein can also be used to treat angiogenesis-mediated disorders such as various forms of arthritis, including rheumatoid arthritis. In these methods, treatment with combinations of the compounds described herein with other agents useful for treating the disorders, such as cyclooxygenase-2 (COX-2) inhibitors, which are well known to those of skill in the art.

The blood vessels in the synovial lining of the joints can undergo angiogenesis. The endothelial cells form new vascular networks and release factors and reactive oxygen species that lead to pannus growth and cartilage destruction. These factors are believed to actively contribute to rheumatoid arthritis and also to osteoarthritis. Chondrocyte activation by angiogenic-related factors contributes to joint destruction, and also promotes new bone formation. The methods described herein can be used as a therapeutic intervention to prevent bone destruction and new bone formation.

Pathological angiogenesis is also believed to be involved with chronic inflammation. Examples of disorders that can be treated using the compounds, compositions and methods described herein include ulcerative colitis, Crohn's disease, bartonellosis, and atherosclerosis.

II. Methods of Promoting Angiogenesis

It is often desirable to promote angiogenesis, particularly to assist in wound healing, or to provide vascularization to occluded vessels or organs or tissue where insufficient vascularization exists. Compounds that promote angiogenesis can be used to treat conditions of vascular insufficiency, including ischemic heart disease, peripheral vascular disease, thromboembolic disease, stroke and vasculititis (Buerger's disease, Wegener's granulomatosis, and Giant Cell Arteritis). Such compounds can also be used at wound sites to promote healing, and at sites of transplantation and grafting (e.g., skin grafting). Spinal cord injuries can also be expected to benefit from intervention of vascularization.

On the cellular level, angiogenesis can be promoted by binding a suitable compound (for example, antibodies, antibody fragments and/or small molecules) to angiostatin in a manner that inhibits the ability of angiostatin to bind to NDPK, provided that the angiostatin, once bound, does not bind to NDPK. The methods involve administering to a patient in need of treatment thereof an effective, angiogenesis-promoting amount of an angiostatin-binding compound. An effective, angiogenesis-promoting amount of such compounds is defined herein as an amount sufficient to promote angiogenesis in a patient. The amount of such compounds, and the duration of treatment, can be readily determined by a treating physician, for example, by monitoring blood flow or other signs of increased vascularization at a desired location in a patient.

Compounds and compositions useful in the angiogenesis-inhibiting and angiogenesis-promoting methods are described in more detail below.

III. Compounds for Promoting or Inhibiting Angiogenesis

Various compounds, including various antibodies, can bind to NDPK and inhibit angiogenesis. Various other compounds can bind to angiostatin. However, the mere fact that they bind to angiostatin does not determine their ultimate effect on angiogenesis, since the compounds, once bound, may not inhibit the ability of angiostatin to bind to NDPK (i.e., the compounds are bound to angiostatin at a site different from where angiostatin binds to NDPK).

The activity of the compounds once bound can be readily determined using the assays described herein. The compounds described herein are not limited to a particular molecular weight. In some cases, large compounds such as antibodies can be preferred since their effect is mostly steric, and therefore will not likely inhibit the function of NDPK systemically, only on the surface of vascular endothelial cells. In other cases, small molecules may be easier to produce in commercial quantities and may be provided in relatively larger doses. The compounds can be large molecules (i.e., those with a molecular weight above about 1000) or small molecules (i.e., those with a molecular weight below about 1000). Examples of suitable types of compounds include antibodies, antibody fragments, enzymes, peptides and oligonucleotides.

A. Antibodies

Antibodies can be generated that bind to NDPK or angiostatin. Polyclonal antibodies can be used, provided their overall effect is a desired effect (i.e., an angiogenic or an anti-angiogenic effect, as desired). However, monoclonal antibodies are preferred. Humanized (chimeric) antibodies can be even more preferred.

The antibodies may not and need not bind to NDPK in exactly the same way as angiostatin. Angiostatin has several potential binding portions (possibly involving the various kringles), and the antibodies likely do not include portions that mimic each of these binding portions.

Antibodies, in particular, monoclonal antibodies (mAbs) can be developed against NDPK that can be used either to directly inhibit angiogenesis or to target cytotoxic drugs or radioisotopic or other labels to sites of angiogenesis. Because angiogenesis does not occur to a large extent in adults, except following tissue injury, the antibodies can be extremely specific. Furthermore, unlike other lines of research which have produced cancer cell-specific mAbs to target cytotoxic drugs to tumors, these mAbs are prepared against host antigens (i.e., NDPK). This approach has the major advantage that generation of “resistant” variants of the tumor cannot occur and, in theory, one mAb can be used to treat all solid tumors. An additional advantage is that endothelial cells, by virtue of their vascular location, are very accessible to antibodies in the circulation.

Antibody Preparation

The term “antibody” refers to a polypeptide substantially encoded by an immunoglobulin gene or immunoglobulin genes, or fragments thereof, that specifically binds and recognizes an analyte (antigen, in this case NDPK or angiostatin). Immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit includes a tetramer. Each tetramer is composed of two identical pairs of polypeptide chains, each pair having one “light” (about 25 kD) and one “heavy” chain (about 50-70 kD). The N-terminus of each chain has a variable region of about 100 to 110 or more amino acids primarily responsible for antigen recognition. The terms “variable light chain” (or “VL”) and “variable heavy chain” (or “VH”) refer to these light and heavy chains, respectively.

Antibodies exist, for example, as intact immunoglobulins or as a number of well characterized antigen-binding fragments produced by digestion with various peptidases. For example, pepsin digests an antibody below the disulfide linkages in the hinge region to produce an F(ab′)2 fragment, a dimer of Fab which itself is a light chain joined to VH-CH1 by a disulfide bond. The F(ab′)2 fragment can be reduced under mild conditions to break the disulfide linkage in the hinge region, thereby converting the F(ab′)2 dimer into an Fab′ monomer. The Fab′ monomer is essentially an Fab with part of the hinge region (see Fundamental Immunology, Third Edition, W. E. Paul (ed.), Raven Press, N.Y. (1993), the contents of which are hereby incorporated by reference). While various antibody fragments are defined in terms of the digestion of an intact antibody, one of ordinary skill in the art will appreciate that such fragments can be synthesized de novo either chemically or by using recombinant DNA methodology. Thus, the term antibody, as used herein, also includes antibody fragments, such as a single chain antibody, an antigen binding F(ab′)2 fragment, an antigen binding Fab′ fragment, an antigen binding Fab fragment, an antigen binding Fv fragment, a single heavy chain or a chimeric (humanized) antibody. Such antibodies can be produced by modifying whole antibodies or synthesized de novo using recombinant DNA methodologies.

The NDPK or angiostatin (including fragments, derivatives, and analogs thereof) can be used as an immunogen to generate antibodies which immunospecifically bind such immunogens. Such antibodies include but are not limited to polyclonal antibodies, monoclonal antibodies, chimeric antibodies, single chain antibodies, antigen binding antibody fragments (e.g., Fab, Fab′, F(ab′)2, Fv, or hypervariable regions), and mAb or Fab expression libraries. In some embodiments, polyclonal and/or monoclonal antibodies to the NDPK or angiostatin are produced. In yet other embodiments, fragments of the NDPK or angiostatin that are identified as immunogenic are used as immunogens for antibody production.

Various procedures known in the art can be used to produce polyclonal antibodies. Various host animals (including, but not limited to, rabbits, mice, rats, sheep, goats, camels, and the like) can be immunized by injection with the antigen, fragment, derivative or analog. Various adjuvants can be used to increase the immunological response, depending on the host species. Such adjuvants include, for example, Freund's adjuvant (complete and incomplete), mineral gels such as aluminum hydroxide, surface active substances such as lysolecithin, pluronic polyols, polyanions, peptides, oil emulsions, keyhole limpet hemocyanins, dinitrophenol, and other adjuvants, such as BCG (bacille Calmette-Guerin) and Corynebacterium parvum.

Any technique that provides for the production of antibody molecules by continuous cell lines in culture can be used to prepare monoclonal antibodies directed toward the NDPK or angiostatin. Such techniques include, for example, the hybridoma technique originally developed by Kohler and Milstein (see, e.g., Nature 256:495-97 (1975)), the trioma technique (see, e.g., Hagiwara and Yuasa, Hum. Antibodies Hybridomas 4:15-19 (1993); Hering et al., Biomed. Biochim. Acta 47:211-16 (1988)), the human B-cell hybridoma technique (see, e.g., Kozbor et al., Immunology Today 4:72 (1983)), and the EBV-hybridoma technique to produce human monoclonal antibodies (see, e.g., Cole et al., In: Monoclonal Antibodies and Cancer Therapy, Alan R. Liss, Inc., pp. 77-96 (1985)). Human antibodies can be used and can be obtained by using human hybridomas (see, e.g., Cote et al., Proc. Natl. Acad. Sci. USA 80:2026-30 (1983)) or by transforming human B cells with EBV virus in vitro (see, e.g., Cole et al., supra).

“Chimeric” or “humanized” antibodies (see, e.g., Morrison et al., Proc. Natl. Acad. Sci. USA 81:6851-55 (1984); Neuberger et al., Nature 312:604-08 (1984); Takeda et al., Nature 314:452-54 (1985)) can also be prepared. Such chimeric antibodies are typically prepared by splicing the non-human genes for an antibody molecule specific for antigen together with genes from a human antibody molecule of appropriate biological activity. It can be desirable to transfer the antigen binding regions (e.g., Fab′, F(ab′)2, Fab, Fv, or hypervariable regions) of non-human antibodies into the framework of a human antibody by recombinant DNA techniques to produce a substantially human molecule. Methods for producing such “chimeric” molecules are generally well known and described in, for example, U.S. Pat. Nos. 4,816,567; 4,816,397; 5,693,762; and 5,712,120; PCT Patent Publications WO 87/02671 and WO 90/00616; and European Patent Publication EP 239 400 (the disclosures of which are incorporated by reference herein). Alternatively, a human monoclonal antibody or portions thereof can be identified by first screening a cDNA library for nucleic acid molecules that encode antibodies that specifically bind to NDPK, in particular, to the H1 subunit thereof, according to the method generally set forth by Huse et al. (Science 246:1275-81 (1989)), the contents of which are hereby incorporated by reference. The nucleic acid molecule can then be cloned and amplified to obtain sequences that encode the antibody (or antigen-binding domain) of the desired specificity. Phage display technology offers another technique for selecting antibodies that bind to NDPK, fragments, derivatives, subunits (in particular, the H1 subunit) or analogs thereof. (See, e.g., International Patent Publications WO 91/17271 and WO 92/01047; Huse et al., supra.)

Techniques for producing single chain antibodies (see, e.g., U.S. Pat. Nos. 4,946,778 and 5,969,108) can also be used. An additional aspect of the invention utilizes the techniques described for the construction of a Fab expression library (see, e.g., Huse et al., supra) to allow rapid and easy identification of monoclonal Fab fragments with the desired specificity for antigens, fragments, derivatives, or analogs thereof.

Antibodies that contain the idiotype of the molecule can be generated by known techniques. For example, such fragments include but are not limited to, the F(ab′)2 fragment which can be produced by pepsin digestion of the antibody molecule, the Fab′ fragments which can be generated by reducing the disulfide bridges of the F(ab′)2 fragment, the Fab fragments which can be generated by treating the antibody molecule with papain and a reducing agent, and Fv fragments. Recombinant Fv fragments can also be produced in eukaryotic cells using, for example, the methods described in U.S. Pat. No. 5,965,405 (the disclosure of which is incorporated by reference herein).

Antibody screening can be accomplished by techniques known in the art (e.g., ELISA (enzyme-linked immunosorbent assay)). In one example, antibodies that recognize a specific domain of an antigen can be used to assay generated hybridomas for a product which binds to polypeptides containing that domain. Antibodies specific to a domain of an antigen are also provided.

Antibodies against the NDPK or angiostatin (including fragments, derivatives and analogs) can be used for passive antibody treatment, according to methods known in the art. The antibodies can be produced as described above and can be polyclonal or monoclonal antibodies and administered intravenously, enterally (e.g., as an enteric coated tablet form), by aerosol, orally, transdermally, transmucosally, intrapleurally, intrathecally, or by other suitable routes.

Small amounts of humanized antibody can be produced in a transient expression system in CHO cells to establish that they bind to NDPK or angiostatin. Stable cell lines can then be isolated to produce larger quantities of purified material.

The binding affinity of murine and humanized antibodies can be determined using the procedure described by Krause et al., Behring Inst. Mitt., 87:56-67 (1990). Briefly, antibodies can be labeled with fluorescein using fluorescein isothiocyanate (FITC), and then incubated with HUVEC cells for two hours on ice in PBS containing fetal calf serum (FCS) and sodium azide. The amount of fluorescence bound per cell can be determined in a FACScan and calibrated using standard beads. The number of molecules of antibody that had bound per cell at each antibody concentration can be established and used to generate Scatchard plots. Competition assays can be performed by FACScan quantitation of bound antibody after incubating the cells with a standard quantity of the murine antibody together with a dilution series of the humanized variants.

High Throughput Screening Methods for mAb Libraries

High throughput monoclonal antibody assays can be used to determine the binding affinities of the antibodies to the subunits, and also identify which antibodies bind to NDPK or angiostatin. The assays can evaluate, for example, increased or decreased ATP levels or the degree of cellular proliferation. Suitable assays are described, for example, in the Examples. Similar high throughput assays can be used to evaluate the properties of small molecule libraries.

Similar screening methods can be used to identify other classes of compounds useful in the methods described herein. Combinatorial libraries of compounds, for example, phage display peptide libraries, small molecule libraries and oligonucleotide libraries can be screened. Compounds that bind to the NDPK or angiostatin can be identified, for example, using competitive binding studies. The effect of the compounds once bound to the NDPK or angiostatin can be determined, for example, by evaluating the level of ATP synthesis, the proliferation of human vascular endothelial cells (HUVEC) and/or the viability and/or growth of tumors.

Antibody/Drug Conjugates

Antibodies raised against NDPK or angiostatin, and, in particular, monoclonal antibodies, can be conjugated to a drug. The drug/antibody complex can then be administered to a patient, and the antibody will bind to the NDPK or angiostatin in a manner that delivers a relatively high concentration of the drug to the desired tissue or organ. In some embodiments, the binding of the drug to the antibody is in a biodegradable linkage, so that the drug is released over time. In other embodiments, the drug remains attached to the antibody.

Anti-cancer drugs are an example of drugs that can be conjugated to the antibodies. For example, the antibodies can be conjugated with QFA, which is an antifolate, or with calicheamicin, adriaicin, bleomicin or vincamicin, which are anti-tumor antibiotics that cleave the double-stranded DNA of tumor cells. Additional tumor-treating compounds that can be coupled to the antibodies include BCNU, streptozoicin, vincristine, ricin, radioisotopes, and 5-fluorouracil and other anti-cancer nucleosides.

In vivo xenograft studies can be used to show that tumor inhibition with limited normal tissue damage can be obtained with antibodies conjugated to these anti-cancer drugs. The antibody/drug conjugates can be used to target compounds directly to tumors that might otherwise be too toxic when administered systemically.

The conjugates are most advantageously used in combination with targeted drug delivery methods, for example, by placing the compounds in liposomes or other microparticles of an appropriate size such that they lodge in capillary beds around tumors and release the compounds at the tumor site. Alternatively the compounds can be injected directly into or around the site of a tumor, for example, via injection or catheter delivery. Such methods minimize any undesirable systemic effects.

Oligonucleotides with free, reactive hydroxy, amine, carboxy or thiol groups at either the 3′ or 5′ end can be conjugated to free reactive groups on antibodies using conventional coupling chemistry, for example, using heterobifunctional reagents such as SPDP. The 3′ or 5′ end of the oligonucleotide can be enzymatically labeled, for example, with 32P as tracer for DNA. Purification can be accomplished, for example, with protein A chromatography. The final product can be tested for cell-binding activity and protein and bound oligonucleotide concentrations. Depending on the activity of the oligonucleotides, the conjugates can be used for therapeutic or diagnostic purposes.

The antibodies (or other compounds that bind to the NDPK or angiostatin) can be conjugated with photosensitizers such as porphyrins and used in targeted photodynamic therapy. After the compositions are administered and allowed to bind to the NDPK or angiostatin in vascular cells, the photodynamic therapy can be conducted by irradiation with light at a suitable wavelength for a suitable amount of time.

Antibodies that bind to the NDPK or angiostatin can also be covalently or ionically coupled to various markers, and used to detect the presence of tumors. This generally involves administering a suitable amount of the antibody to the patient, waiting for the antibody to bind to the NDPK or angiostatin at or around a tumor site, and detecting the marker. Suitable markers are well known to those of skill in the art, and include for example, radioisotopic labels, fluorescent labels and the like, and detection methods for these markers are also well known to those of skill in the art. Examples of suitable detection techniques include positron emission tomography, autoradiography, flow cytometry, radioreceptor binding assays, and immunohistochemistry.

Generally, a background concentration of the compounds will be observed in locations throughout the body. However, a higher, detectable concentration will be observed in locations where a tumor is present. The label can be detected, and, accordingly, the tumors can be detected.

Multivalent Compounds

Multivalent compounds are defined herein as compounds that include more than one moiety capable of being attached to NDPK and/or angiostatin. Preferably, at least one moiety binds to NDPK.

In one embodiment, the multifunctional compound includes at least one protein and/or peptide chain. Alternatively, the compound can include small molecules with a plurality of moieties with bind properties as described above.

B. Small Molecules

As used herein, small molecules are defined as molecules with molecular weights below about 2000, except in the case of oligonucleotides that can be considered small molecules if their molecular weight is less than about 10,000 (about 30 mer or less). Many companies currently generate libraries of small molecules, and high throughput screening methods for evaluating small molecule libraries to identify compounds that bind particular receptors are well known to those of skill in the art. Combinatorial libraries of small molecules can be screened and suitable compounds for use in the methods described herein can be identified using routine experimentation. One example of a suitable small molecule library is a phage display library. Another such library is a library including random oligonucleotides, typically with sizes less than about 100 mers. The SELEX process can be used to screen such oligonucleotide libraries (including DNA, RNA and other types of genetic material, and also including natural and non-natural base pairs) for compounds that have suitable binding properties, and other assays can be used to determine the effect of the compounds on angiogenesis.

The SELEX method is described in U.S. Pat. No. 5,270,163 to Gold et al. Briefly, a candidate mixture of single stranded nucleic acids with regions of randomized sequence can be contacted with NDPK and those nucleic acids having an increased affinity to NDPK can be partitioned from the remainder of the candidate mixture. The partitioned nucleic acids can be amplified to yield a ligand enriched mixture.

C. Peptide Phage Display Libraries

One technique that is useful for identifying peptides that bind to NDPK or angiostatin is phage-display technology, as described, for example, in Phage Display of Peptides and Proteins: A Laboratory Manual; Edited by Brian K. Kay et al. Academic Press San Diego, 1996, the contents of which are hereby incorporated by reference for all purposes.

Phage peptide libraries typically include numerous different phage clones, each expressing a different peptide, encoded in a single-stranded DNA genome as an insert in one of the coat proteins. In an ideal phage library the number of individual clones would be 20ⁿ, where “n” equals the number of residues that make up the random peptides encoded by the phage. For example, if a phage library was screened for a seven residue peptide, the library in theory would contain 20⁷ (or 1.28×10⁹) possible 7-residue sequences. Therefore, a 7-mer peptide library should contain approximately 10⁹ individual phage.

Methods for preparing libraries containing diverse populations of various types of molecules such as peptides, polypeptides, proteins, and fragments thereof are known in the art and are commercially available (see, for example, Ecker and Crooke, Biotechnology 13:351-360 (1995), and the references cited therein, the contents of each of which is incorporated herein by reference for all purposes). One example of a suitable phage display library is the Ph.D.-7 phage display library (New England BioLabs Cat #8100), a combinatorial library consisting of random peptide 7-mers. The Ph.D.-7 phage display library consists of linear 7-mer peptides fused to the pIII coat protein of M13 via a Gly-Gly-Gly-Ser flexible linker. The library contains 2.8×109 independent clones and is useful for identifying targets requiring binding elements concentrated in a short stretch of amino acids.

Phage clones displaying peptides that are able to bind to the NDPK or angiostatin are selected from the library. The sequences of the inserted peptides are deduced from the DNA sequences of the phage clones. This approach is particularly desirable because no prior knowledge of the primary sequence of the target protein is necessary, epitopes represented within the target, either by a linear sequence of amino acids (linear epitope) or by the spatial juxtaposition of amino acids distant from each other within the primary sequence (conformational epitope) are both identifiable, and peptidic mimotopes of epitopes derived from non-proteinaceous molecules such as lipids and carbohydrate moieties can also be generated.

A library of phage displaying potential binding peptides can be incubated with immobilized NDPK or angiostatin to select clones encoding recombinant peptides that specifically bind the immobilized NDPK or angiostatin. The phages can be amplified after various rounds of biopanning (binding to the immobilized NDPK or angiostatin) and individual viral plaques, each expressing a different recombinant protein, or binding peptide, can then be expanded to produce sufficient amounts of peptides to perform a binding assay.

Phage selection can be conducted according to methods known in the art and according to manufacturers' recommendations. The “target” proteins, NDPK or angiostatin, can be coated overnight onto tissue culture plates in humidified containers. In a first round of panning, approximately 2×10¹¹ phage can be incubated on the protein-coated plate for 60 minutes at room temperature while rocking gently. The plates can then be washed using standard wash solutions. The binding phage can then be collected and amplified following elution using the target protein. Secondary and tertiary pannings can be performed as necessary.

Following the last screening, individual colonies of phage-infected bacteria can be picked at random, the phage DNA isolated and then subjected to automated dideoxy sequencing. The sequence of the displayed peptides can be deduced from the DNA sequence.

IV. Compositions

Therapeutic, prophylactic and diagnostic compositions containing the compounds described herein typically include one or more active compounds together with a pharmaceutically acceptable excipient, diluent or carrier for in vivo use. Such compositions can be readily prepared by mixing the active compound(s) with the appropriate excipient, diluent or carrier.

Any suitable dosage may be administered. The type of angiogenesis-mediated disorder to be treated (cancer, rheumatoid arthritis, and the like), the compound, the carrier and the amount will vary widely depending on body weight, the severity of the condition being treated and other factors that can be readily evaluated by those of skill in the art. Generally a dosage of between about 1 milligrams (mg) per kilogram (kg) of body weight and about 100 mg per kg of body weight is suitable.

A dosage unit may include a single compound or mixtures thereof with other compounds or other anti-cancer agents, if the composition is used to treat cancer, or other anti-arthritic agents, such as COX-2 inhibitors, if the composition is used to treat rheumatoid arthritis. The dosage unit can also include diluents, extenders, carriers and the like. The unit may be in solid or gel form such as pills, tablets, capsules and the like or in liquid form suitable for oral, rectal, topical, intravenous injection or parenteral administration or injection into or around the tumor

The compounds are typically mixed with a pharmaceutically acceptable carrier. This carrier can be a solid or liquid and the type is generally chosen based on the type of administration being used.

The compounds can be administered via any suitable route of administration that is effective in the treatment of the particular angiogenesis-mediated disorder that is being treated. Treatment may be oral, rectal, topical, parenteral or intravenous administration or by injection into the tumor and the like. The method of administering an effective amount also varies depending on the angiogenesis-mediated disorder being treated. It is believed that parenteral treatment by intravenous, subcutaneous, or intramuscular application of the compounds, formulated with an appropriate carrier, additional cancer inhibiting compound or compounds or diluents to facilitate administration, will be the preferred method of administering the compounds.

The compounds can be incorporated into a variety of formulations for therapeutic administration. More particularly, the compounds can be formulated into pharmaceutical compositions by combination with appropriate, pharmaceutically acceptable carriers or diluents, and may be formulated into preparations in solid, semi-solid, liquid or gaseous forms, such as tablets, capsules, pills, powders, granules, dragees, gels, slurries, ointments, solutions, suppositories, injections, inhalants and aerosols. As such, administration of the compounds can be achieved in various ways, including oral, buccal, rectal, parenteral, intraperitoneal, intradermal, transdermal, intracheal, etc., administration. Moreover, the compounds can be administered in a local rather than systemic manner, for example via injection of the compound directly into a solid tumor, often in a depot or sustained release formulation. In addition, the compounds can be administered in a targeted drug delivery system, for example, in a liposome coated with the antibodies described herein. Such liposomes will be targeted to and taken up selectively by the tumor.

In addition, the compounds can be formulated with common excipients, diluents or carriers, and compressed into tablets, or formulated as elixirs or solutions for convenient oral administration, or administered by the intramuscular or intravenous routes. The compounds can be administered transdermally, and can be formulated as sustained release dosage forms and the like.

The compounds can be administered alone, in combination with each other, or they can be used in combination with other known compounds (e.g., other anti-cancer drugs or other drugs, such as anti-inflammatories, antibiotics, corticosteroids, vitamins, etc.). For instance, the compounds can be used in conjunctive therapy with other known anti-angiogenic chemotherapeutic or antineoplastic agents (e.g., vinca alkaloids, antibiotics, antimetabolites, platinum coordination complexes, etc.). For instance, the compounds can be used in conjunctive therapy with a vinca alkaloid compound, such as vinblastine, vincristine, taxol, etc.; an antibiotic, such as adriamycin (doxorubicin), dactinomycin (actinomycin D), daunorubicin (daunomycin, rubidomycin), bleomycin, plicamycin (mithramycin) and mitomycin (mitomycin C), etc.; an antimetabolite, such as methotrexate, cytarabine (AraC), azauridine, azaribine, fluorodeoxyuridine, deoxycoformycin, mercaptopurine, etc.; or a platinum coordination complex, such as cisplatin (cis-DDP), carboplatin, etc. In addition, the compounds can be used in conjunctive therapy with other known anti-angiogenic chemotherapeutic or antineoplastic compounds. In pharmaceutical dosage forms, the compounds may be administered in the form of their pharmaceutically acceptable salts, or they may also be used alone or in appropriate association, as well as in combination with other pharmaceutically active compounds.

Suitable formulations for use in the present invention are found in Remington's Pharmaceutical Sciences (Mack Publishing Company, Philadelphia, Pa., 17th ed. (1985)), which is incorporated herein by reference. Moreover, for a brief review of methods for drug delivery, see, Langer, Science 249:1527-1533 (1990), which is incorporated herein by reference. The pharmaceutical compositions described herein can be manufactured in a manner that is known to those of skill in the art, i.e., by means of conventional mixing, dissolving, granulating, dragee-making, levigating, emulsifying, encapsulating, entrapping or lyophilizing processes. The following methods and excipients are merely exemplary and are in no way limiting.

For injection, the compounds can be formulated into preparations by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. Preferably, the compounds can be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining with pharmaceutically acceptable carriers that are well known in the art. Such carriers enable the compounds to be formulated as tablets, pills, dragees, capsules, emulsions, lipophilic and hydrophilic suspensions, liquids, gels, syrups, slurries, suspensions and the like, for oral ingestion by a patient to be treated. Pharmaceutical preparations for oral use can be obtained by mixing the compounds with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas, or from propellant-free, dry-powder inhalers. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds are preferably formulated for parenteral administration by injection, e.g., by bolus injection or continuous infusion. Formulations for injection may be presented in unit dosage form, e.g., in ampules or in multidose containers, with an added preservative. The compositions may take such forms as suspensions, solutions or emulsions in oily or aqueous vehicles, and may contain formulator agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter, carbowaxes, polyethylene glycols or other glycerides, all of which melt at body temperature, yet are solidified at room temperature.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Alternatively, other delivery systems for hydrophobic pharmaceutical compounds may be employed. Liposomes and emulsions are well known examples of delivery vehicles or carriers for hydrophobic drugs. In a presently preferred embodiment, long-circulating, i.e., stealth, liposomes are employed. Such liposomes are generally described in Woodle, et al., U.S. Pat. No. 5,013,556, the contents of which are hereby incorporated by reference.

The compounds can be encapsulated in a vehicle such as liposomes that facilitates transfer of the bioactive molecules into the targeted tissue, as described, for example, in U.S. Pat. No. 5,879,713 to Roth et al., the contents of which are hereby incorporated by reference. The compounds can be targeted by selecting an encapsulating medium of an appropriate size such that the medium delivers the molecules to a particular target. For example, encapsulating the compounds within microparticles, preferably biocompatible and/or biodegradable microparticles, which are appropriate sized to infiltrate, but remain trapped within, the capillary beds and alveoli of the lungs can be used for targeted delivery to these regions of the body following administration to a patient by infusion or injection.

In a preferred embodiment, the liposome or microparticle has a diameter which is selected to lodge in particular regions of the body. For example, a microparticle selected to lodge in a capillary will typically have a diameter of between 10 and 100, more preferably between 10 and 25, and most preferably, between 15 and 20 microns. Numerous methods are known for preparing liposomes and microparticles of any particular size range. Synthetic methods for forming gel microparticles, or for forming microparticles from molten materials, are known, and include polymerization in emulsion, in sprayed drops, and in separated phases. For solid materials or preformed gels, known methods include wet or dry milling or grinding, pulverization, classification by air jet or sieve, and the like.

Microparticles can be fabricated from different polymers using a variety of different methods known to those skilled in the art. The solvent evaporation technique is described, for example, in E. Mathiowitz, et al., J. Scanning Microscopy, 4, 329 (1990); L. R. Beck, et al., Fertil. Steril., 31, 545 (1979); and S. Benita, et al., J. Pharm. Sci., 73, 1721 (1984). The hot-melt microencapsulation technique is described by E. Mathiowitz, et al., Reactive Polymers, 6, 275 (1987). The spray drying technique is also well known to those of skill in the art. Spray drying involves dissolving a suitable polymer in an appropriate solvent. A known amount of the compound is suspended (insoluble drugs) or co-dissolved (soluble drugs) in the polymer solution. The solution or the dispersion is then spray-dried. Microparticles ranging between 1-10 microns are obtained with a morphology which depends on the type of polymer used.

Microparticles made of gel-type polymers, such as alginate, can be produced through traditional ionic gelation techniques. The polymers are first dissolved in an aqueous solution, mixed with barium sulfate or some bioactive agent, and then extruded through a microdroplet forming device, which in some instances employs a flow of nitrogen gas to break off the droplet. A slowly stirred (approximately 100-170 RPM) ionic hardening bath is positioned below the extruding device to catch the forming microdroplets. The microparticles are left to incubate in the bath to allow sufficient time for gelation to occur. Microparticle particle size is controlled by using various size extruders or varying either the nitrogen gas or polymer solution flow rates.

Particle size can be selected according to the method of delivery which is to be used, typically IV injection, and where appropriate, entrapment at the site where release is desired.

Liposomes are available commercially from a variety of suppliers. Alternatively, liposomes can be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound or its monophosphate, diphosphate, and/or triphosphate derivatives are then introduced into the container. The container is then swirled by hand to free lipid material from the sides of the container and to disperse lipid aggregates, thereby forming the liposomal suspension.

The monoclonal antibodies specific for NDPK or angiostatin as described herein can optionally be conjugated to liposomes and the delivery can be targeted in this manner. In addition, targeting of a marker on abnormal tumor vasculature can be employed. The targeting moiety when coupled to a toxic drug or radioisotope will act to concentrate the drug where it is needed. Ligands for tumor-associated vessel markers can also be used. For example, a cell adhesion molecule that binds to a tumor vascular element surface marker can be employed. Liposomes and other drug delivery systems can also be used, especially if their surface contains a ligand to direct the carrier preferentially to the tumor vasculature. Liposomes offer the added advantage of shielding the drug from most normal tissues. When coated with polyethylene glycol (PEG) (i.e., stealth liposomes) to minimize uptake by phagocytes and with a tumor vasculature-specific targeting moiety, liposomes offer longer plasma half-lives, lower non-target tissue toxicity, and increased efficacy over non-targeted drug. Using the foregoing methods, the compounds can be targeted to the tumor vasculature to effect control of tumor progression or to other sites of interest (e.g., endothelial cells).

Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various types of sustained-release materials have been established and are well known by those skilled in the art. Sustained-release capsules may, depending on their chemical nature, release the compounds for a few days up to over 100 days. Such sustained release capsules typically include biodegradable polymers, such as polylactides, polyglycolides, polycaprolactones and copolymers thereof.

Pharmaceutical compositions suitable for use in the methods described herein include compositions wherein the active ingredients are contained in a therapeutically effective amount. The amount of composition administered will, of course, be dependent on the subject being treated, on the subject's weight, the severity of the affliction, the manner of administration and the judgment of the prescribing physician. Determination of an effective amount is well within the capability of those skilled in the art, especially in light of the detailed disclosure provided herein.

Therapeutically effective dosages for the compounds described herein can be estimated initially from cell culture assays. For example, a dose can be formulated in animal models to achieve a circulating concentration range that includes the IC50 as determined in cell culture (i.e., the concentration of test compound that is lethal to 50% of a cell culture), or the IC 100 as determined in cell culture (i.e., the concentration of compound that is lethal to 100% of a cell culture). Such information can be used to more accurately determine useful doses in humans. Initial dosages can also be estimated from in vivo data.

Moreover, toxicity and therapeutic efficacy of the compounds described herein can be determined by standard pharmaceutical procedures in cell cultures or experimental animals, e.g., by determining the LD50, (the dose lethal to 50% of the population) and the ED50 (the dose therapeutically effective in 50% of the population). The dose ratio between toxic and therapeutic effect is the therapeutic index and can be expressed as the ratio between LD50 and ED50. Compounds which exhibit high therapeutic indices are preferred. The data obtained from these cell culture assays and animal studies can be used in formulating a dosage range that is not toxic for use in human. The dosage of such compounds lies preferably within a range of circulating concentrations that include the ED50 with little or no toxicity. The dosage may vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics, Ch. 1, p. 1).

Dosage amount and interval may be adjusted individually to provide plasma levels of the active compound which are sufficient to maintain therapeutic effect. Preferably, therapeutically effective serum levels will be achieved by administering multiple doses each day. In cases of local administration or selective uptake, the effective local concentration of the drug may not be related to plasma concentration. One having skill in the art will be able to optimize therapeutically effective local dosages without undue experimentation.

While the composition may be administered by routes other than intravenously (i.v.), intraveneous administration is preferred. This is because the target of the therapy is primarily the proliferating vasculature comprising the angiogenesis; and thus, administering the composition intravenously saturates the targeted vasculature much quicker than if another route of administration is used. Additionally, the intravenous route allows for the possibility of further targeting to specific tissues.

In one embodiment, a catheter is used to direct the composition directly to the location of the target angiogenesis. For example, if tumor angiogenesis is the target of the anti-angiogenic therapy, and if the tumor is located in the liver, then the immunoconjugate or the unconjugated antibody or a fragment thereof may be delivered into the hepatic portal vein using a catheter. In this embodiment, systemic distribution of composition is minimized, further minimizing any potential side effects from the antiangiogenic therapy.

V. Screening Methods

Various screening methods can be used to determine the ability of compounds to inhibit the binding of angiostatin to NDPK or to bind to NDPK. In the methods described herein, compounds can bind to a position on angiostatin and inhibit angiostatin binding to NDPK or can directly bind to NDPK. The mere fact that a compound binds to angiostatin does not determine its ultimate effect on angiogenesis.

Various other screening methods can also be used to determine the activity of compounds bound to the NDPK or angiostatin. Examples of suitable screening methods include measuring ATP synthesis and measuring the cellular proliferation of human vascular endothelial cells (HUVEC).

The compounds can be evaluated using in vitro assays to determine their biological activity. These assays are familiar to those skilled in the art and include HUVEC and BCE proliferation assays, HUVEC wound/migration assay, endothelial cell tube forming assay, CAM assay, MatrigelTM invasion assay and the rat aortic assay. The ability of a compound to inhibit or promote angiogenesis in these assays would indicate that the compound is able to mimic or inhibit the interaction of angiostatin with NDPK.

The biological activity of the compounds may also be tested in vivo. Examples of suitable assays include the B16B16 metastasis assay or the Lewis Lung Carcinoma primary tumor or metastasis assays. In such experiments, the activity of the compounds can be compared to that of angiostatin if desired.

Suitable binding assays are described in more detail below.

VI. Binding Assays

The structure of NDPK is known. The entire NDPK molecule can be used in the present assays or a subunit thereof can be used, as can a fusion protein comprising the NDPK, the subunit thereof or the angiostatin-binding domain thereof. The binding assays described herein can use any such truncated forms of the NDPK. A preferred subunit is the H1 subunit.

Binding assays include cell-free assays in which NDPK or angiostatin is incubated with a test compound (proteinaceous or non-proteinaceous) which, advantageously, bears a detectable label (e.g., a radioactive or fluorescent label). Following incubation, the NDPK or angiostatin, free or bound to test compound, can be separated from unbound test compound using any of a variety of techniques. For example, the NDPK or angiostatin can be bound to a solid support (e.g., a plate or a column) and washed free of unbound test compound. The amount of test compound bound to NDPK or angiostatin, is then determined, for example, using a technique appropriate for detecting the label used (e.g., liquid scintillation counting and gamma counting in the case of a radiolabeled test compound or by fluorometric analysis).

Binding assays can also take the form of cell-free competition binding assays. In such assays, NDPK or angiostatin is incubated with a compound known to interact with NDPK or angiostatin, which compound, advantageously, bears a detectable label (e.g., a radioactive or fluorescent label). A test compound (proteinaceous or non-proteinaceous) is added to the reaction and assayed for its ability to compete with the known (labeled) compound for binding to NDPK or angiostatin.

Free known (labeled) compound can be separated from bound known compound, and the amount of bound known compound determined to assess the ability of the test compound to compete. This assay can be formatted so as to facilitate screening of large numbers of test compounds by linking the NDPK or angiostatin to a solid support so that it can be readily washed free of unbound reactants. A plastic support, for example, a plastic plate (e.g., a 96 well dish), is preferred. NDPK or angiostatin suitable for use in the cell-free assays described above can be isolated from natural sources (e.g., membrane preparations) or prepared recombinantly or chemically. The NDPK or angiostatin can be prepared as a fusion protein using, for example, known recombinant techniques. Preferred fusion proteins include a GST (glutathione-S-transferase) moiety, a GFP (green fluorescent protein) moiety (useful for cellular localization studies) or a His tag (useful for affinity purification). The non-NDPK or angiostatin moiety can be present in the fusion protein N-terminal or C-terminal to the NDPK or angiostatin.

As indicated above, the NDPK (or H1 subunit thereof or angiostatin binding domain thereof) or angiostatin can be present on the surface of a cell, in purified form, or linked to a solid support, including a plastic or glass plate or bead, a chromatographic resin (e.g., Sepharose), a filter or a membrane. Methods for attaching proteins to such supports are well known in the art and include direct chemical attachment and attachment via a binding pair (e.g., biotin and avidin or biotin and streptavidin). Whether free or bound to a solid support, the NDPK or angiostatin can be unlabeled or can bear a detectable label (e.g., a fluorescent or radioactive label).

A test compound identified in one or more of the above-described assays as being capable of binding to NDPK or angiostatin can, potentially, promote or inhibit angiogenesis, cellular migration, proliferation and pericellular proteolysis and, potentially, inhibit the ability of angiostatin to bind NDPK. To determine the specific effect of any particular test compound selected on the basis of its ability to bind NDPK or angiostatin, assays can be conducted to determine, for example, the effect of various concentrations of the selected test compound on activity, for example, cell (e.g., endothelial cell) proliferation, metabolism or cytosolic/cytoplasmic pH. (Assays can be conducted to determine the effect of test compounds on NDPK or angiostatin activity using standard enzyme assay protocols.)

Cell proliferation can be monitored by measuring uptake of labeled bases into cellular nucleic acids, for example, radioactively (e.g.,³H, SiC, ¹⁴C), fluorescently (e.g., CYQUANT (Molecular Probes)) or colorimetrically (e.g., BrdU (Boehringer Mannheim or MTS (Promega)). Cytosolic/cytoplasmic pH determinations can be made with a digital imaging microscope using substrates such as BCECF (bis(carboxyethyl)-carbonyl fluorescein) (Molecular Probes, Inc.).

A test compound that reduces or replaces the concentration of angiostatin required to inhibit cellular proliferation or lower intracellular pH can be expected to do so by acting as NDPK-binding compound. A test compound that enhances cellular proliferation in the presence of angiostatin (or functional portion thereof or functional equivalent thereof) can be expected to do so by binding to angiostatin in a manner that inhibits the binding of angiostatin to NDPK.

A test compound that raises or lowers intracellular pH in the presence of angiostatin (or functional portion thereof or functional equivalent thereof) may do so by binding to NDPK. These functional assays can be conducted in the absence of angiostatin (i.e., test compound alone), with angiostatin (or functional portion thereof or functional equivalent thereof) run as a separate control. A test compound that, for example, modulates intracellular pH in the absence of angiostatin can itself bind to NDPK.

Other types of assays that can be carried out to determine the effect of a test compound on angiostatin binding to NDPK or angiostatin include the Lewis Lung Carcinoma assay (O'Reilly et al., Cell 79:315 (1994)) and extracellular migration assays (Boyden Chamber assay: Kleinman et al., Biochemistry 25:312 (1986) and Albini et al., Can. Res. 47:3239 (1987)). Accordingly, the methods permit the screening of compounds for their ability to modulate the effect of angiostatin on ATP synthesis as modulated by NDPK.

In addition to the various approaches described above, assays can also be designed so as to be monitorable colorometrically or using time-resolved fluorescence.

In another embodiment, the invention relates to compounds identified using the above-described assays as being capable of binding to NDPK or angiostatin. Such compounds can include novel small molecules (e.g., organic compounds (for example, organic compounds less than 500 Daltons), and novel polypeptides, oligonucleotides, as well as novel natural products (preferably in isolated form) (including alkyloids, tannins, glycosides, lipids, carbohydrates and the like). Compounds that bind to the NDPK can be used to inhibit angiogenesis, for example, in tumor bearing patients and in patients suffering from vascular related retinopathies (including diabetic) and Terigium.

The compounds identified in accordance with the above assays can be formulated as pharmaceutical compositions.

VII. Kits

Kits suitable for conducting the assays described herein can be prepared. Such kits can include NDPK and/or angiostatin. These components can bear a detectable label. The kit can include an NDPK or angiostatin-specific antibody. Plasminogen can also be present.

The kit can include any of the above components disposed within one or more container means. The kit can further include ancillary reagents (e.g., buffers) for use in the assays. Diagnostic methods based on the assays for binding angiostatin to NDPK can be used to identify patients suffering from angiogenesis-mediated disorders. The demonstration that NDPK is an angiostatin binding protein, and the resulting availability of methods of identifying agents that can be used to modulate the effects of angiostatin, make it possible to determine which individuals will likely be responsive to particular therapeutic strategies. Treatment strategies for individuals suffering from angiogenesis-mediated disorders can be designed more effectively and with greater predictability of a successful result. Thus, for a given angiogenesis-mediated disorder that is of polygenic (non-Mendelian) origin, one would select that genotype that is implicated not only in the disease, but also in that variant of the disease that is associated with abnormal angiogenesis and proceed to screen, via a diagnostic procedure, all future patients having the same genotype in order to choose that therapeutic strategy most associated with a successful outcome or least associated with a toxic side effect, for that genotype.

All documents cited above are hereby incorporated in their entirety by reference.

From the foregoing, it will be obvious to those skilled in the art that various modifications in the above-described methods, and compositions can be made without departing from the spirit and scope of the invention. Accordingly, the invention may be embodied in other specific forms without departing from the spirit or essential characteristics thereof. Present embodiments and examples, therefore, are to be considered in all respects as illustrative and not restrictive, and all changes which come within the meaning and range of equivalency of the claims are therefore intended to be embraced therein.

We claim:

1. A composition for use in inhibiting angiogenesis by binding to NDPK comprising:
a) a compound that binds to NDPK, and
b) a suitable carrier.

2. The composition of claim 1, wherein the compound binds to the H1 subunit of NDPK.

3. The composition of claim 1, wherein the compound that binds to NDPK is selected from the group consisting of antibodies, antibody fragments, enzymes, peptides and oligonucleotides.

4. The composition of claim 1, wherein the compound that binds to NDPK is a conjugate of an anti-tumor agent that does not bind to the NDPK and a compound that does bind to NDPK.

5. The composition of claim 1, wherein the compound that binds to NDPK is an antibody or an antibody fragment.

6. The composition of claim 5, wherein the antibody is a monoclonal antibody.

7. The composition of claim 5, wherein the antibody is a humanized antibody.

8. The composition of claim 1, wherein the compound that binds to NDPK is present in or conjugated onto a liposome or microparticle that is of a suitable size for intraveneous administration but that lodges in capillary beds.

9. The composition of claim 1, further comprising an anti-tumor agent that does not bind to NDPK.

10. The composition of claim 1, further comprising a COX-2 inhibitor.

11. The composition of claim 1, further comprising an angiogenesis-promoting agent that does not bind to NDPK.

12. A method of inhibiting angiogenesis, comprising administering to a patient in need of treatment thereof an effective, angiogenesis inhibiting amount of a compound that binds to NDPK.

13. The method of claim 12, wherein the compound that binds to NDPK is angiostatin.

14. The method of claim 12, wherein the compound that binds to NDPK is selected from the group consisting of antibodies, antibody fragments, enzymes, peptides and oligonucleotides.

15. The method of claim 12, wherein the compound that binds to NDPK is an antibody or an antibody fragment.

16. The method of claim 15, wherein the antibody is a monoclonal antibody.

17. The method of claim 16, wherein the antibody is a humanized antibody.

18. The method of claim 12, wherein the compound that binds to NDPK is present in or conjugated onto a liposome or microparticle that is of a suitable size for intraveneous administration but that lodges in capillary beds.

19. The method of claim 12, further comprising administering an anti-tumor agent that does not bind to NDPK.

20. The method of claim 12, further comprising administering a COX-2 inhibitor.

21. The method of claim 12, wherein the compound that binds to NDPK is administered intravenously or intramuscularly.

22. A method of promoting angiogenesis, comprising administering to a patient in need of treatment thereof an effective, angiogenesis-promoting amount of a compound that binds to angiostatin in a manner that inhibits the ability of angiostatin to bind to NDPK.

23. The method of claim 22, wherein the compound is selected from the group consisting of antibodies, antibody fragments, enzymes, peptides, and oligonucleotides.

24. The method of claim 22, wherein the compound is a conjugate of an angiogenesis-promoting compound that does not bind to angiostatin and a compound that does bind to angiostatin in a manner that inhibits the ability of angiostatin to bind to NDPK.

25. The method of claim 22, wherein the compound is an antibody or antibody fragment.

26. The method of claim 25, wherein the antibody is a monoclonal antibody.

27. The method of claim 25, wherein the antibody is a humanized antibody.

28. The method of claim 22, wherein the compound is present in or conjugated to a liposome or microparticle that is of a suitable size for intraveneous administration but that lodges in capillary beds.

29. The method of claim 22, further comprising administering an angiogenesis-promoting agent that does not bind to angiostatin.

30. The method of claim 22, wherein the compound is administered intravenously or intramuscularly.

31. The method of claim 22, wherein the compound is administered locally to a location in a patient in need of increased vascularization.

32. A method of screening a test compound for its ability to inhibit the binding of angiostatin to NDPK comprising:
i) contacting the test compound and angiostatin with NDPK under conditions such that angiostatin can bind to the NDPK in the absence of the test compound, and
ii) determining the amount of angiostatin bound to the subunits, and comparing that amount to an amount of angiostatin bound to the subunits in the absence of the test compound, wherein a reduction in the amount of angiostatin bound to the NDPK in the presence of the test compound indicates that the test compound inhibits the binding of angiostatin to the NDPK, and wherein an increase of the amount of angiostatin bound to the NDPK in the presence of the test compound indicates that the test compound enhances the binding of angiostatin to the NDPK.

33. The method of claim 32 wherein the angiostatin bears a detectable label.

34. The method of claim 32 wherein the NDPK is attached to a solid support.

35. The method of claim 32 wherein the NDPK is associated with a lipid membrane.

36. The method of claim 35 wherein the membrane is a membrane of an intact cell.

37. A compound identified in the method of claim 32 as inhibiting the binding of angiostatin to NDPK.

38. A monoclonal antibody specific for NDPK that functions as an angiogenesis inhibitor.

39. A monoclonal antibody specific for angiostatin that functions as an angiogenesis promoter.

40. A method of screening a test compound for its ability to inhibit angiogenesis via binding to NDPK comprising:
i) contacting the test compound with NDPK under conditions such that angiostatin would bind to the NDPK in the absence of the test compound, and
ii) determining the binding affinity of the compound to NDPK and/or iii) performing one or more bioassays to determine the amount of angiogenesis mediated by the NDPK bound to the test compound.
